(19) United States
(12) Patent Application Publication
(10) Pub. No.: US 2015/0299743 A1
(43) Pub. Date: Oct. 22, 2015

(54) PROCESS FOR PRODUCTION OF AN ALKYL METHACRYLATE

(71) Applicant: LUCITE INTERNATIONAL UK LIMITED, Southampton, Hampshire (GB)

(72) Inventors: Graham Ronald Eastham, Redcar (GB); David William Johnson, Redcar (GB); Marco Wilhemus Fraaije, Nijenborgh (NL); Remko Tsjibbe Winter, Nijenborgh (NL)

(21) Appl. No.: 14/653,567

(22) PCT Filed: Dec. 20, 2013
(86) PCT No.: PCT/GB2013/053385

§ 371 Date: Jun. 18, 2015

(30) Foreign Application Priority Data
Dec. 21, 2012 (GB) 1223271.6

(51) Int. Cl. C12P 7/62; C08F 120/10

(52) U.S. Cl. CPC C12P 7/62; C08F 120/10

(57) ABSTRACT

A process for the production of an alkyl methacrylate, particularly methyl methacrylate, is provided, in which a Baeyer-Villiger Monooxygenase enzyme is used to convert an alkylisopropenylketone substrate to the relevant alkyl methacrylate by abnormal asymmetric oxygen insertion. The invention provides a biobased route to key industrial monomers in particular for the generation of polymers such as poly methyl methacrylate.

The present invention relates to a process for production of an alkyl methacrylate from an alkylisopropenylketone by the use of a novel enzyme catalysed process, and polymers and copolymers produced therefrom.

Acrylic acids and their alkyl esters, in particular, Methacrylic acid (MAA) and its methyl ester, methyl methacrylate (MMA) or ethyl ester, ethyl methacrylate (EMA) are important monomers in the chemical industry. Their main application is in the production of plastics for various applications. The most significant polymerisation application is the casting, moulding or extrusion of polymethyl methacrylate (PMMA) or polyethyl methacrylate (PEMA) to produce high optical clarity plastics. In addition, many copolymers are used, important copolymers are copolymers of methyl methacrylate and ethyl methacrylate with α-methyl styrene, ethyl acrylate and butyl acrylate. Furthermore, by a simple transesterification reaction, MMA and EMA may be converted to other esters such as butyl methacrylate, lauryl methacrylate etc.

Currently MMA (and MAA) is produced by a number of chemical procedures, one of which is the successful ‘Alpha process’ whereby MMA is obtained from the ester methyl propionate by anhydrous reaction with formaldehyde. In the Alpha process, the methyl propionate is produced by the carbonylation of ethylene. This ethylene feedstock is derived from fossil fuels. Recently, it has become desirable to also source sustainable biomass feedstocks for the chemical industry. Accordingly, an alternative biomass route to MMA and instead of using the alpha process would be advantageous.

Currently EMA is produced by a number of chemical procedures, one of which is the direct esterification of methacrylic acid; another is the transesterification of MMA with ethyl acetate.

Therefore it is one object of the present invention to solve the aforementioned problem, and provide a biological or part biological process for the production of alkyl methacrylates.

Surprisingly, the present inventors have found a way to apply unusual enzymes in a novel process to form alkyl methacrylates at an industrially applicable level, thereby providing a new and viable bio-based route to key monomers such as MMA and EMA.

According to a first aspect of the present invention there is provided a process of producing an alkyl methacrylate comprising the steps of;
(i) converting an alkylisopropenylketone to the corresponding alkyl methacrylate using a Baeyer-Villiger monooxygenase enzyme.

The above process may further comprise the step of formation of an alkylisopropenylketone from raw feedstocks, wherein the term ‘raw feedstocks’ includes any base organic chemical capable of being transformed into an alkylisopropenylketone, for example an alkylisopropylketone, a ketone, a carboxylic acid or an alcohol.

In addition, the above process may further comprise the step of performing one or more chemical, biochemical or biological conversions to produce raw feedstocks, wherein the term ‘raw feedstocks’ is as defined hereinbefore.

As used herein, the term ‘corresponding’ with reference to converting an alkylisopropenylketone to the relevant alkyl methacrylate means the alkyl group of the alkyl methacrylate produced is the same as the alkyl group of the starting alkylisopropenylketone and the methacrylate is the acyloxy product of the isopropenylketone acyl group.

Preferably the alkylisopropenylketone used in the above process is methylisopropenylketone or ethylisopropenylketone. More preferably the alkylisopropenylketone is methylisopropenylketone, which ketone may be selected and combined with any of the aspects, embodiments, or other preferred features of the present invention as contained herein.

In one preferred embodiment, when the alkylisopropenylketone is methylisopropenylketone, suitably the alkyl methacrylate produced is methyl methacrylate which methacrylate may be combined with any of the aspects, embodiments, or other preferred features of the present invention as contained herein.

Therefore, according to a preferred embodiment of a first aspect of the present invention there is provided a process of producing methyl methacrylate comprising the steps of;

(i) converting methylisopropenylketone to methyl methacrylate by abnormal oxidation using a Baeyer-Villiger monooxygenase enzyme

Methylisopropenylketone may be prepared by any suitable chemical, biochemical or biological method known in the art.

An example of a suitable chemical method of preparing methylisopropenylketone is the reaction of 2-butanone with formaldehyde or a derivative thereof as described in, for example, U.S. Pat. No. 5,072,051, U.S. Pat. No. 3,422,148, or U.S. Pat. No. 5,637,774.

U.S. Pat. No. 5,072,051 describes in the examples listed in column 4 from line 28 the reaction of methylethylketone (2-butanone) with paraformaldehyde in the presence of a secondary amine hydrohalide and catalysed by a carboxylic acid of up to 15 carbon atoms, for example propionic acid, or a solid non soluble oxide of an element of group IB, IIIA, IVA, VA, VB, VIB, and VIII, for example niobium oxide. Preferably the reaction is performed as described in U.S. Pat. No. 5,072,051 at 135° C. for one hour with stirring under 400-800 kPa then increasing to 700-1400 kPa of pressure, the reaction mixture comprising relative molar amounts of 1 methylethylketone, 0.25 paraformaldehyde, 0.25 secondary amine hydrohalide, 0.001 hydroquinone, and 0.01 catalyst.

U.S. Pat. No. 3,422,148 describes in example 1 the reaction of methylethylketone and aqueous formaldehyde catalysed by an acid cation exchanger, for example a sulfonated styrene-divinyl benzene polymer. Preferably the reaction is performed as described by U.S. Pat. No. 3,422,148 in a reaction tube heated with steam, at 130 C. at a pressure of 15 atm, the reaction tube comprising 560 cc of catalyst, and the reaction mixture comprising methylethylketone with 30 wt % aqueous formaldehyde at a molar ratio of 6:1.

U.S. Pat. No. 5,637,774 describes in example 3, the reaction of methylethylketone and aqueous formalin catalysed by an acidic zeolite catalyst, specifically in example 1, a 5 Angstrom zeolite catalyst exchanged with ammonium and calcined. Preferably the reaction is performed by charging a reaction tube with 75 cc of catalyst and maintaining a flow of nitrogen at 180-230 cc per minute over the catalyst during the course of the reaction. Preferably the reaction mixture comprises 81.6 wt % methylethylketone, 6.8 wt % formaldehyde, and 11.6 wt % water passed through the reaction tube at a temperature of 330° C. for 4.2-12.8 seconds.

A further example of a suitable alternative chemical method of preparing methylisopropenylketone is the reaction of methylisopropylketone as described in, for example, U.S. Pat. No. 4,146,574.

U.S. Pat. No. 4,146,574 describes in application number 5 of the examples, the oxidative degradation of methylisopropylketone. Preferably the reaction is performed by passing a mixed gas of methylisopropylketone (10.9), water (52.8), oxygen (15.0) and nitrogen (234.9) through a reaction tube comprising 2 ml of catalyst 1 (values for the reaction mixture given in mmol/hr). Catalyst 1 preferably comprising 10 ml of aqueous heteropolyphosphoric acid dried onto 3 g of diatomaceous earth carrier as prepared in example 1.

Therefore, according to a further preferred embodiment of a first aspect of the present invention, there is provided a process of producing methyl methacrylate comprising the steps of:
(i) production of methylisopropenylketone from raw feedstocks; and
(ii) converting the methylisopropenylketone produced to methyl methacrylate using a Baeyer-Villiger monooxygenase enzyme.

Preferably step (i) is performed by any of the methods described above.

The raw feedstocks may be prepared by any suitable chemical, biochemical or biological method known in the art.

Preferably the raw feedstocks include 2-butanone, methylisopropylketone, and formaldehyde or a derivative thereof.

Examples of suitable biological methods of preparing 2-butanone include the reaction of 2-butanol with an alcohol dehydrogenase enzyme suitably under EC group 1.1.1.X, or the reaction of acetoin with an alcohol dehydrogenase enzyme suitably under EC group 1.1.1.X to produce 2.3-butandiol which is reacted with a diol dehydratase enzyme suitably under EC group 4.2.1.X, or the reaction of acetoin with an alcohol dehydratase enzyme suitably under EC group 4.2.1.X to produce methylvinylketone which is reacted with an enone reductase enzyme suitably under EC group number 1.1.1.X or 1.3.1.X.

The biological method of reaction of 2-butanol with an alcohol dehydrogenase enzyme suitably under EC group 1.1.1.X, may be performed using an alcohol dehydrogenase enzyme of EC number 1.1.1.1 or 1.1.1.2 from any suitable organism.

The alternate biological method of reaction of acetoin with an alcohol dehydrogenase enzyme suitably under EC group 1.1.1.X to produce 2.3-butandiol which is reacted with a diol dehydratase enzyme suitably under EC group 4.2.1.X, may be performed using an alcohol dehydrogenase enzyme of EC group 1.1.1.4 or 1.1.1.76 and a diol dehydratase enzyme under EC group 4.2.1.28 from any suitable organism.

The alternate biological method of reaction of acetoin with an alcohol dehydratase enzyme suitably under EC group 4.2.1.X to produce methylvinylketone which is reacted with an enone reductase enzyme suitably under EC group number 1.1.1.X or 1.3.1.X, may be performed using an enzyme that can act as an alcohol dehydratase under EC group number 4.2.1.53 or 4.2.1.43 or 4.2.1.3 or an enzyme described by Jianfeng et al. in Chemical Communications, 2010, 46, 8588-8590 and using an enzyme capable of reducing methylvinylketone such as those enzymes under EC group number 1.1.1.54 or 1.3.1.31 or those described by Yamamoto et al. in U.S. Pat. No. 6,780,967.

According to a further preferred embodiment of a first aspect of the present invention, there is provided a process a process of producing methyl methacrylate comprising the steps of;
(i) production of methylisopropenylketone from 2-butanone; and
(ii) converting the methylisopropenylketone to methyl methacrylate by abnormal oxidation using a Baeyer-Villiger monooxygenase enzyme.

According to a further preferred embodiment of a first aspect of the present invention, there is provided a process a process of producing methyl methacrylate comprising the steps of;
(i) production of 2-butanone from 2-butanol and/or acetoin;
(ii) production of methylisopropenylketone from the 2-butanone; and
(iii) converting the methylisopropenylketone to methyl methacrylate by abnormal oxidation using a Baeyer-Villiger monooxygenase enzyme.

According to a further preferred embodiment of a first aspect of the present invention, there is provided a process a process of producing methyl methacrylate comprising the steps of;
(i) production of 2-butanone from 2-butanol and/or acetoin by one or more of the following routes:
   a. from 2-butanol with an alcohol dehydrogenase enzyme under EC group 1.1.1.X; and/or
   b. from acetoin with an alcohol dehydrogenase enzyme under EC group 1.1.1.X to produce 2.3-butandiol which is reacted with a diol dehydratase enzyme under EC group 4.2.1.X; and/or
   c. from acetoin with an alcohol dehydratase enzyme under EC group 4.2.1.X to produce methylvinylketone which is reacted with an enone reductase enzyme under EC group number 1.1.1.X or 1.3.1.X;
(ii) production of methylisopropenylketone from the 2-butanone; and
(iii) converting the methylisopropenylketone to methyl methacrylate by abnormal oxidation using a Baeyer-Villiger monooxygenase enzyme.

Suitable methods used to perform such enzymatic transformations are well known in the art, however examples of enzyme sources and descriptions of how to use them for the above transformations are contained within our corresponding patent application GB1209425.6.

Examples of suitable chemical methods of preparing 2-butanone include dehydrogenation of 2-butanol, or the oxidation of 1 or 2-butene, or the oxidation of isobutylbenzene, or isolation from the oxygenate stream of the liquid phase oxidation of naphtha, or isolation from the oxygenate stream of the Fischer-Tropsch reaction.

The dehydrogenation of 2-butanol, may be performed by any method known in the art, suitable reaction conditions include using a catalyst of one of copper, silver, zinc or bronze held on a basic support such as silica or alumina at temperatures of between 190 to 280 C. and at pressures of 1 atm. For example, see the experimental of ‘The dehydrogenation of 2-butanol over copper-based catalysts: optimising catalyst composition and determining kinetic parameters’ by Keuler et al. Applied Catalysis A: General 218 (2001) pp 171-180.

The oxidation of 1 or 2-butene, may be performed by any method known in the art, suitable reactions conditions include using a palladium (II) salt catalyst, specifically a halide free mixture of palladium and copper salts with a heterpolyanion dissolved in aqueous acetonitrile at 75-85 C. and under 5 atm of oxygen pressure as per the Wacker process. For example, U.S. Pat. No. 5,557,014 example 57 describes a two stage homogeneous catalytic route using palladium salts and phosphomolydovanadates for oxidation of 1-butene to 2-butanone. U.S. Pat. No. 5,506,363 example 68 shows a similar system.

Isolation from the oxygenate stream of the liquid phase oxidation of naphtha, or isolation from the oxygenate stream of the Fischer-Tropsch reaction, may be performed by any method known in the art, suitably by fractionation of the mixed oxygenate streams as described in, for example U.S. Pat. No. 4,686,317 or Ashford's Dictionary of Industrial Chemicals, Third Edition, 2011, page 6013.

Examples of suitable methods of preparing methylisopropylketone include any known chemical, biochemical or biological processes known in the art.

Preferably formaldehyde or derivative thereof is selected from 1,1 dimethoxymethane, higher formals of formaldehyde and methanol, CH₃—O—(CH₂—O)ᵣ—CH₃ where i=2, formalin or a mixture comprising formaldehyde, methanol and methyl propionate.

Preferably, by the term formalin is meant a mixture of formaldehyde:methanol:water in the ratio 25 to 65%:0.01 to 25%:25 to 70% by weight. More preferably, by the term formalin is meant a mixture of formaldehyde:methanol:water in the ratio 30 to 60%:0.03 to 20%:35 to 60% by weight. Most preferably, by the term formalin is meant a mixture of formaldehyde:methanol:water in the ratio 35 to 55%:0.05 to 18%:42 to 53% by weight.

Examples of suitable methods of preparing formaldehyde include the reaction of methanol and air using a silver powder or an iron molybdate based catalyst as described in US Environmental Protection Agency Document EPA-450/4-91-012 “Locating and Estimating Emissions from Sources of Formaldehyde (revised)”, March 1991.

Optionally, the raw feedstocks and/or any of the reactants necessary to make them as defined in the exemplary methods above may be sourced from biomass. More preferably, at least one of the raw feedstocks or the reactants necessary for production thereof is sourced from biomass.

Therefore, according to a further preferred embodiment of a first aspect of the present invention, there is provided a process of producing methyl methacrylate comprising the steps of:
(i) production of raw feedstocks from biomass;
(ii) production of alkylisopropenylketone from the raw feedstocks; and
(iii) converting the alkyklisopropenylketone produced to methyl methacrylate by abnormal oxidation using a Baeyer-Villiger monooxygenase enzyme.

Suitable methods for preparing base organic chemicals, such as those used herein as raw feedstocks, from biomass are well known in the art. By way of example, 2-butanone may be produced from biomass via 2,3-butandiol, the 2,3-butandiol having been produced from fermentation of sugar containing biomass by microorganisms. Suitable microorganisms that may be used to produce 2,3-butandiol are, for example, Bacillus polymyxa, Lactobacillus brevis or Klebsiella pneumoniae. Conversion of the 2,3-butandiol produced into 2-butanone may be performed by any known method, for example dehydration by catalysis with morden bentonite clays at temperatures lower than 350 C. Any sugar containing biomass may be used, for example any lignocellulosic, or starch based biomass. For example, see the method described in ‘Bulk Chemicals from Biomass’ by van Havaren et al. Biofuels, Bioprod. Bioref. 2:41-57 (2008).

In another preferred embodiment, the alkylisopropenylketone is ethylisopropenylketone, and the alkyl methacrylate produced is ethyl methacrylate.

Ethylisopropenylketone may be prepared by any suitable chemical, biochemical or biological method known in the art.

An example of a suitable chemical method of preparing ethylisopropenylketone is the reaction of 3-propanone (diethylketone) with formaldehyde or derivatives thereof as described in, for example, ‘Copolymerisation of alkylisopropenylketone with styrene’ by Kinoshita et al. Journal of Polymer Science 21, 5, pp 359-366, or ‘Interchange of Functionality on Conjugated Carbonyl Compounds through Isoxazoles’ by Buechi et al. J.Am.Chem.Soc., 1972, 94 (26), pp 9128-9132 with reference to ‘Process for the preparation of αβ-dialcoylglycerols’ by Colonge et al. Bull. Soc. Chim. Fr., 838 (1947).

Kinoshita et al. describe in the experimental section of the paper, the formation of various alkylisopropenylketones by the reaction of the corresponding alkylethylketone and paraformaldehyde in the presence of dimethyl amine hydrochloride and ethanol under reflux conditions, with the addition of hydrochloric acid over 10 hours. The mixture is then cooled and the relevant alkylisopropenylketone product isolated by thermal decomposition.

Buechi et al. describe in the experimental section 1e, the formation of isopropenyl ethyl ketone by the condensation of diethyl ketone and formaldehyde as described by Colonge et al. Colonge describes under section 1 the general reaction of an aliphatic ketone with formaldehyde, then under section 2 the formation of alpha ethylenic ketones wherein the formation of 2-methyl-pent-1-ene-3-one is described from the condensation of alkaline formaldehyde with ethylenic derivatives of methylpropylketone over a Rayney nickel catalyst.

Therefore, according to a further preferred embodiment of a first aspect of the present invention, there is provided a process of producing ethyl methacrylate comprising the steps of:
(i) production of ethylisopropenylketone from raw feedstocks;
(ii) converting the ethylisopropenylketone produced to methyl methacrylate using a Baeyer-Villiger monooxygenase enzyme.

Preferably step (i) is performed by any of the methods described above.

The raw feedstocks may be prepared by any suitable chemical, biochemical or biological method known in the art.

Preferably the raw feedstocks include 3-pentanone, ethylisopropylketone and formaldehyde or derivatives thereof.

An example of a suitable biological method of preparing 3-pentanone is the reaction of 3-pentanol with an alcohol dehydrogenase enzyme suitably under EC group 1.1.1.X.

The reaction of 3-pentanone with an alcohol dehydrogenase enzyme suitably under EC group 1.1.1.X, may be performed using an alcohol dehydrogenase enzyme of EC number 1.1.1.1 or 1.1.1.2 from any suitable organism.

Suitable methods used to perform such an enzymatic transformation are well known in the art, however suitable examples are given in ‘Substrate specificity and stereoselectivity of horse liver alcohol dehydrogenase’ by Adolph et al. Eur. J. Biochem. 201 (1991) pp 615-625, or ‘TADH, the thermostable alcohol dehydrogenase from Thermus sp. ATN1: a versatile new biocatalyst for organic synthesis' by Hollrigl et al. Appl. Microbiol. Biotechnol. 2008 November; 81(2):263-73.

Examples of suitable chemical methods of preparing 3-pentanone include the ketonization of propionic acid, the dehydrogenation of 1-propanol, or the hydrocarbonylation of ethene.

The ketonization of propionic acid, may be performed by any method known in the art, such as passing propionic acid vapour over a zirconium oxide catalyst at 350° C. as described in U.S. Pat. No. 4,574,074 example 7. An alternative suitable reaction includes using 2.4 g of Platinum impregnated with niobium oxide as a catalyst at 523K for 4 hours under flowing hydrogen at 80 cm3/minute with a 40% propionic acid solution passing over at pressure of 825 psi. For example, see ‘Catalytic upgrading of biomass-derived acids by dehydration/hydrogenation and C—C coupling reactions’ by Serrano-Ruiz et al. University of Wisconsin. Further suitable catalysts include cerium (IV) oxide or manganese dioxide on an alumina support, metal oxide catalysts derived from bulk Keggin heteropoly acids (HPA) H₃₊ₙ[PMo₁₂₋ₙVₙO₄₀] (n=0-2) or a Caesium salt thereof in the vapour phase at 350° C. and 1 bar hydrogen pressure in a fixed bed reactor, or stable polyoxometalate H₃PW₁₂O₄₀ (HPW) supported on a silica or a bulk acidic salt Cs_{2.5}H_{0.5}PW₁₂O₄₀ (CsPW) support at 250-300 C. in flowing hydrogen and nitrogen gas. For example, see Deoxygenation of Biomass-Derived Molecules over Multifunctional Polyoxometalate Catalysts in the Gas Phase′ by Alotaibi et al. University of Liverpool.

The dehydrogenation of 1-propanol, may be performed by any method known in the art, suitable reaction conditions include using 0.15 g of a CeO2-Fe2O3 catalyst at 450 C. in a nitrogen down flow of 73 mmolh with 1-propanol passed over at a rate of 23 mmolh. For example, see ‘Synthesis of 3-pentanone from 1-propanol over CeO2-Fe2O3 catalysts’ by Kamimura et al. Applied Catalysis A: General 252 (2003) 399-410.

The hydrocarbonylation of ethene, may be performed by any method known in the art, suitable reaction conditions include using an aqueous trifluoroacetic acid solution of Pd(OAc)2/PPh3 under mild conditions in the presence of a 2:1:1 mixture of ethene:carbon monoxide:hydrogen/water. For example, see ‘The production of low molecular weight oxygenates from carbon monoxide and ethene’ by Robertson et al. Coordination Chemistry Reviews 225 (2002) 67-90.

Examples of suitable methods of preparing ethylisopropylketone include any chemical, biochemical or biological method known in the art.

Methods of preparation of formaldehyde or derivatives thereof have been described above.

Optionally, the raw feedstocks and/or any of the reactants necessary to make them as defined in the exemplary methods above may be sourced from biomass. More preferably, at least one of the raw feedstocks or the reactants necessary for production thereof is sourced from biomass.

Suitable methods for preparing base organic chemicals, such as those used herein as raw feedstocks, from biomass are well known in the art. By way of example, ethene may be produced from biomass via ethanol which is produced during fermentation of sugar containing biomass. The bio-ethanol produced can be converted by well known techniques such as the dehydration reaction at 300-600 C. over any catalyst selected from alumina, activated clay, zeolite, or mordenite into ethene. Any sugar containing biomass may be used, for example any lignocellulosic, or starch based biomass, but preferably the biomass used is high in sugars such as sugar beet or sugarcane. For example, see the method described in ‘Bulk Chemicals from Biomass’ by van Havaren et al. Biofuels, Bioprod. Bioref. 2:41-57 (2008).

As defined in accordance with the process of the first aspect of the present invention, once the alkylisopropenylketone is formed, it is converted to the relevant alkyl methacrylate by the action of a Baeyer-Villiger monooxygenase enzyme.

Baeyer-Villiger oxidation refers to the insertion of an oxygen atom into a ketone to form an ester. In asymmetric ketones, this insertion reaction occurs almost exclusively between the carbonyl carbon and the most stable carbonium ion of the ketone. It is generally known that Baeyer-Villiger oxy-insertion for unsymmetrical ketones has the approximate order of migration of tertiary alkyl> secondary alkyl, aryl> primary alkyl> methyl group (March's Advanced Organic Chemistry: Reactions, Mechanisms and Structure 6^{th} Edition, pg. 1619). Accordingly, Baeyer-Villiger oxidation of an alkylisopropenylketone would traditionally be associated with the product of an isopropenylester. Therefore, Baeyer Villiger oxidation of an alkylisopropenylketone is not a route that the skilled person would readily choose as a route to alkyl methacrylates such as MMA or EMA. Nevertheless, the inventors have found that Baeyer-Villiger monooxygenases can insert an oxygen atom into alkylisopropenylketones in an abnormal manner, yielding the unlikely product of alkyl methacrylates. Furthermore the use of alkylisopropenylketones to produce alkyl methacrylates in itself is not a well explored route in terms of chemical processing, and the authors are not aware of any analogous industrial chemical process currently in use. Accordingly, firstly using alkylisopropenylketones as a starting material to produce alkyl methacrylates is in itself unusual, and secondly seeking a bio-based enzymatic version of such a route is unprecedented.

Surprisingly, therefore, this has led to an unusual and novel biological route to alkyl methacrylate monomers for the polymer industry via abnormal Baeyer-Villiger oxidation.

By the term ‘abnormal’ when used herein in relation to the oxidation of an alkylisopropenylketone substrate by a Baeyer Villiger Monooxygenase enzyme, it is meant that the enzyme catalyses the insertion of an oxygen between the carbonyl carbon and the alkyl group of the ketone.

It is known that Baeyer-Villiger oxidative enzymes are common to various organisms including bacteria, plants, animals, archea, and fungi. Baeyer-Villiger oxidative enzymes can catalyse the conversion of ketones to esters. However, those enzymes that are reported are only described as acting in biological systems on ring based ketones (lactones), rather than the straight chain aliphatic ketones. In the few studies where their activity on straight chain aliphatic ketones has been tested, they are reported as having very low activity. There are no reports of the action of Baeyer-Villager oxidative enzymes on unsaturated aliphatic ketones.

The term Baeyer-Villiger monooxygenase′ as used herein preferably refers to an enzyme capable of catalysing oxidation reactions belonging to the EC classification group 1.14.13.X and such enzymes generally comprise the following characteristic sequences: two Rossman fold protein sequence motifs (GxGxxG) at the N-terminus and the middle of the protein sequence respectively, and the typical BVMO binding motif FxGxxxHxxxW[P/D] located in a loop region of the folded protein which characteristic sequences may be selected and combined with any of the aspects, embodiments, or other preferred features of the present invention as contained herein.

According to a further preferred embodiment of the first aspect of the present invention there is provided a process of producing an alkyl methacrylate, preferably, methyl methacrylate, comprising the steps of;
(i) converting an alkylisopropenylketone, preferably methyl isopropenylketone to the corresponding alkyl methacrylate using an enzyme comprising the following characteristic sequences: two Rossman fold protein sequence motifs (GxGxxG) at the N-terminus and the middle of the protein sequence respectively, and the typical BVMO binding motif FxGxxxHxxxW[P/D] located in a loop region of the folded protein

The Baeyer-Villiger monooxygenase may be a wild type enzyme, or a modified enzyme which enzyme type may be selected and combined with any of the aspects, embodiments, or other preferred features of the present invention as contained herein capable of working with a wild type or modified enzyme. In addition, the enzyme may be synthetic whether in accordance with the wild type or a modification thereof which enzyme type may be selected and combined with any of the aspects, embodiments, or other preferred features of the present invention as contained herein capable of working with a synthetic enzyme.

In any case, the modified Baeyer-Villiger monooxygenase should preferably be as active and/or selective as the wild type, more preferably, more active and/or selective than the wild type in the oxidative transformation of the alkylisopropenylketone, preferably, methyl isopropenylketone to produce alkylmethacrylate.

The term ‘wild type’ as used herein whether with reference to polypeptides such as enzymes, polynucleotides such as genes, organisms, cells, or any other matter refers to the naturally occurring form of said matter.

The term ‘modified’ as used herein with reference to polypeptides such as enzymes, polynucleotides such as genes, organisms, cells, or any other matter refers to such matter as being different to the wild type.

The term ‘microbe convertible gas(es)’ as used herein means a gas or gases that can be converted by microbes into a raw feedstock. A suitable gas is a gas rich in CO and a suitable fermentation is described in US 2012/0045807A1 which converts CO to 2,3-butandiol using anaerobic fermentation with Clostridia such as Clostridium autoethanogenum, ljundahlii and ragsdalei in appropriate media and under the conditions known to the skilled person.

Suitable alterations to wild type matter that may produce modified matter include alterations to the genetic material, alterations to the protein material.

Alterations to the genetic material may include any genetic modification known in the art which will render the material different to the wild type.

Examples of such genetic modifications include, but are not limited to: deletions, insertions, substitutions, fusions etc. which may be performed on the polynucleotide/s sequence containing the relevant gene or genes to be modified.

Such genetic modifications within the scope of the present invention may also include any suitable epigenetic modifications. Epigenetic modifications may include any modification that affects the relevant genetic material without modification of the polynucleotide/s sequence containing the relevant gene or genes to be modified. Examples of epigenetic modifications include, but are not limited to; nucleic acid methylation or acetylation, histone modification, paramutation, gene silencing, etc.

Alterations to protein material may include any protein modification known in the art which will render the material different to the wild type.

Examples of such protein modifications include, but are not limited to: cleaving parts of the polypeptide including fragmentation; attaching other biochemically functional groups; changing the chemical nature of an amino acid; changing amino acid residues including conservative and non-conservative substitutions, deletions, insertions etc; changing the bonding of the polypeptide etc; which may be performed on the polypeptide/s sequence which fold(s) to form the relevant protein or proteins to be modified.

Alterations to the structure of said materials may include any structural modification known in the art which will render the structure of genetic or protein material different to the wild type.

Examples of such structural modifications include modifications caused by, but not limited to, the following factors: the interaction with other structures; interactions with solvents; interactions with substrates, products, cofactors, coenzymes, or any other chemical present in a suitable reaction including other polynucleotides or polypeptides; the creation of quaternary protein structures; changing the ambient temperature or pH etc. which may be performed on the structure(s) of the relevant genetic or protein material(s) of interest.

Each of the modifications detailed under the groups of genetic alteration, protein alteration or structural alteration above are given as an exemplification of the wide range of possible modifications known to the skilled man, and are not intended to limit the scope of the present invention.

Preferably the Baeyer-Villiger Monooxygenase (BVMO) enzyme is a wild type enzyme which feature may be combined with any of the aspects, embodiments, or other preferred features of the present invention as contained herein capable of working with a wild type enzyme.

More preferably the Baeyer-Villiger Monooxygenase (BVMO) enzyme is a wild type enzyme deriving from an organism, wherein the organism may be from any domain including the archaea, bacteria or eukarya. Still more preferably the Baeyer-Villiger Monooxygenase (BVMO) enzyme is a wild type enzyme deriving from an organism, wherein the organism is from the kingdom of plants, fungi, archaea or bacteria. Still more preferably the Baeyer-Villiger Monooxygenase (BVMO) enzyme is a wild type enzyme deriving from a bacterium, a fungus, or an archaeon which organisms may be selected and combined with any of the aspects, embodiments, or other preferred features of the present invention as contained herein which are capable of working with a bacterium, fungus or archaeon as applicable.

In one embodiment, the Baeyer-Villiger Monooxygenase (BVMO) enzyme is a wild type enzyme deriving from a bacterium.

In one embodiment, the Baeyer-Villiger Monooxygenase (BVMO) enzyme is a wild type enzyme deriving from a fungus.

In one embodiment, the Baeyer-Villiger Monooxygenase (BVMO) enzyme is a wild type enzyme deriving from an archaeon.

According to a further preferred embodiment of the first aspect of the present invention there is provided a process of producing an alkyl methacrylate comprising the steps of;
(i) converting an alkylisopropenylketone to the corresponding alkyl methacrylate using a wild type Baeyer-Villiger monooxygenase enzyme.

According to a further preferred embodiment of the first aspect of the present invention there is provided a process of producing an alkyl methacrylate comprising the steps of;
(i) converting an alkylisopropenylketone to the corresponding alkyl methacrylate using a wild type Baeyer-Villiger monooxygenase enzyme deriving from a bacterium, fungus or archaeon.

Suitable bacterial sources of wild type Baeyer-Villiger Monooxygenase (BVMO) enzymes include, but are not limited to, bacteria from the following bacterial genera; Acinetobacter, Rhodococcus, Arthrobacter, Brachymonas, Nocardia, Exophiala, Brevibacterium, Gordonia, Novosphingobium, Streptomyces, Thermobifida, Xanthobacter, Mycobacterium, Comamonas, Thermobifida or Pseudomonas which bacterial genera may be selected and combined with any of the aspects, embodiments, or other preferred features of the present invention as contained herein which are capable of working with a bacterium.

Preferred bacterial sources of wild type Baeyer-Villiger monooxygenase (BVMO) enzymes are bacteria from the following genera: Acinetobacter or Rhodococcus.

According to a further preferred embodiment of the first aspect of the present invention there is provided a process of producing an alkyl methacrylate comprising the steps of;
(i) converting an alkylisopropenylketone to the corresponding alkyl methacrylate using a wild type Baeyer-Villiger monooxygenase enzyme deriving from bacteria of the genera Acinetobacter or Rhodococcus.

Suitable fungal sources of wild type Baeyer-Villiger Monooxygenase (BVMO) enzymes include, but are not limited to, fungi from the following fungal genera; Gibberella, Aspergillus, Maganporthe, Cylindrocarpon, Curvularia, Drechslera, Saccharomyces, Candida, Cunninghamella, Cylindrocarpon, or Schizosaccharomyces.

Preferred fungal sources of wild type Baeyer-Villiger monooxygenase (BVMO) enzymes are fungi from the following genera: Gibberella, Aspergillus or Magnaporthe.

Most preferably the Baeyer-Villiger monooxygenase herein is a wild type enzyme deriving from the bacterial species Rhodococcus jostii which bacterial species may be combined with any of the aspects, embodiments, or other preferred features of the present invention as contained herein which are capable of working with a bacterium.

According to a further preferred embodiment of the first aspect of the present invention there is provided a process of producing an alkyl methacrylate comprising the steps of;
(i) converting an alkylisopropenylketone, preferably, methyl isopropenylketone, to the corresponding alkyl methacrylate by abnormal oxidation using a wild type Baeyer-Villiger monooxygenase enzyme deriving from the bacterial species Rhodococcus jostii.

The Baeyer-Villiger monooxygenase may be a type I, type II or type O Baeyer-Villiger monooxygenase, preferably the Baeyer-Villiger monooxygenase is a type I Baeyer-Villiger Monooxygenase.

More preferably the Baeyer-Villiger monooxygenase is a type I Baeyer-Villiger monooxygenase selected from one of the following enzyme groups; a cyclohexanone monooxygenases (CHMO) EC number 1.14.13.22 (GenBank: BAA86293.1); a phenylacetone monooxygenases (PAMO) EC number 1.14.13.92 (Swiss-Prot: Q47PU3); a 4-hydroxyacetophenone monooxygenase (HAPMO) EC number 1.14.13.84 (GenBank: AAK54073.1); an acetone monooxygenases (ACMO) (GenBank: BAF43791.1); a methyl ketone monooxygenases (MEKA) (GenBank: AB115711.1); a cyclopentadecanone monooxygenases (CPDMO) (GenBank: BAE93346.1); a cyclopentanone monooxygenases (CPMO) (GenBank: BAC22652.1); a steroid monooxygenases (STMO) (GenBank: BAA24454.1) which enzyme groups may be selected and combined with any of the aspects, embodiments, or other preferred features of the present invention as contained herein which are capable of working with a type I Baeyer-Villiger monooxygenase

Still more preferably the Baeyer-Villiger monooxygenase is a cyclohexanone monooxygenase, a 4-hydroxyacetophenone monooxygenase, a cyclopentadecanone monooxygenase or an acetone monooxygenase, which may be selected from one of the following enzymes: cyclohexanone monooxygenase from Acinetobacter calcoaceticus NCIMB 9871, cyclohexanone monooxygenases from Xanthobacter flavus (GenBank: CAD10801.1), cyclohexanone monooxygenases from Rhodococcus sp. HI-31 (GenBank: BAH56677.1), cyclohexanone monooxygenase from Rhodococcus jostii RHA1, cyclohexanone monooxygenase from Brachymonas petroleovorans (GenBank: AAR99068.1), 4-hydroxyacetophenone monooxygenase (Q93TJ5.1), cyclopentadecanone monooxygenase (GenBank: BAE93346.1), or acetone monooxygenase from Gordonia sp. TY-5 (Genbank: BAF43791.1).

According to a further preferred embodiment of the first aspect of the present invention there is provided a process of producing an alkyl methacrylate comprising the steps of;
(i) converting an alkylisopropenylketone, preferably, methyl isopropenylketone, to the corresponding alkyl methacrylate by abnormal oxidation using a type I Baeyer-Villiger monooxygenase enzyme.

According to a further preferred embodiment of the first aspect of the present invention there is provided a process of producing an alkyl methacrylate comprising the steps of;
(i) converting an alkylisopropenylketone, preferably, methyl isopropenylketone to the corresponding alkyl methacrylate by abnormal oxidation using a type I Baeyer-Villiger monooxygenase enzyme selected from a cyclohexanone monooxygenase, a 4-hydroxyacetophenone monooxygenase, a cyclopentadecanone monooxygenase or an acetone monooxygenase.

According to a further preferred embodiment of the first aspect of the present invention there is provided a process of producing an alkyl methacrylate comprising the steps of;
(i) converting an alkylisopropenylketone, preferably, methyl isopropenylketone, to the corresponding alkyl methacrylate by abnormal oxidation using a type I Baeyer-Villiger monooxygenase enzyme selected from cyclohexanone monooxygenase from Acinetobacter calcoaceticus NCIMB 9871, cyclohexanone monooxygenases from Xanthobacter flavus (GenBank: CAD10801.1), cyclohexanone monooxygenases from Rhodococcus sp. HI-31 (GenBank: BAH56677.1), cyclohexanone monooxygenase from Rhodococcus jostii RHA1, cyclohexanone monooxygenase from Brachymonas petroleovorans (GenBank: AAR99068.1), 4-hydroxyacetophenone monooxygenase (Q93TJ5.1), cyclopentadecanone monooxygenase (GenBank: BAE93346.1), or acetone monooxygenase from Gordonia sp. TY-5 (Genbank: BAF43791.1)

According to a further preferred embodiment of the first aspect of the present invention there is provided a process of producing an alkyl methacrylate comprising the steps of;
(i) converting an alkylisopropenylketone, preferably, methyl isopropenylketone, to the corresponding alkyl methacrylate by abnormal oxidation using a cyclohexanone monooxygenase (CHMO)

According to a further preferred embodiment of the first aspect of the present invention there is provided a process of producing an alkyl methacrylate comprising the steps of;
(i) converting an alkylisopropenylketone, preferably, methyl isopropenylketone, to the corresponding alkyl methacrylate by abnormal oxidation using a cyclohexanone monooxygenase selected from one of the following enzymes: cyclohexanone monooxygenase from Acinetobacter calcoaceticus NCIMB 9871, cyclohexanone monooxygenases from Xanthobacter flavus (GenBank: CAD10801.1), cyclohexanone monooxygenases from Rhodococcus sp. HI-31 (GenBank: BAH56677.1), cyclohexanone monooxygenase from Rhodococcus jostii RHA1, cyclohexanone monooxygenase from Brachymonas petroleovorans (GenBank: AAR99068.1),

Still more preferably, the Baeyer-Villiger monooxygenase is a cyclohexanone monooxygenase, or an acetone monooxygenase, which may be selected from cyclohexanone monooxygenase from Acinetobacter calcoaceticus NCIMB 9871, cyclohexanone monooxygenases from Xanthobacter flavus (GenBank: CAD10801.1), cyclohexanone monooxygenases from Rhodococcus sp. HI-31 (GenBank: BAH56677.1), cyclohexanone monooxygenase from Rhodococcus jostii RHA1, cyclohexanone monooxygenase from Brachymonas petroleovorans (GenBank: AAR99068.1), or acetone monooxygenase from Gordonia sp. TY-5 (Genbank: BAF43791.1) which enzymes and sources thereof may be selected and combined with any of the aspects, embodiments, or other preferred features of the present invention as contained herein which are capable of working with said enzymes

Most preferably, the Baeyer-Villiger monooxygenase is a cyclohexanone monooxygenase enzyme comprising accession number ro06679 derived from the bacterial species Rhodococcus jostii RHA1 which enzyme and source thereof may be combined with any of the aspects, embodiments, or other preferred features of the present invention as contained herein which are capable of working with said enzyme.

According to a further preferred embodiment of the first aspect of the present invention there is provided a process of producing an alkyl methacrylate comprising the steps of;
(i) converting an alkylisopropenylketone, preferably, methyl isopropenylketone, to the corresponding alkyl methacrylate by abnormal oxidation using a cyclohexanone monooxygenase enzyme comprising accession number ro06679 derived from the bacterial species Rhodococcus jostii RHA1

Optionally, the Baeyer-Villiger Monooxygenase used in the present invention may be present as a mixture of one or more of the abovementioned Baeyer-Villiger Monooxygenase enzymes. In which case, the Baeyer-Villiger Monooxygenase (BVMO) may be derived from any one or more of the sources described above, in any combination or formulation. For example, the Baeyer-Villiger Monooxygenase (BVMO) may be a mixture of a BVMO enzyme derived from a bacterium and a BVMO enzyme derived from a fungus, where one enzyme may be a modified enzyme and one may be a wild type enzyme.

Alternatively, in a further embodiment, the Baeyer-Villiger Monooxygenase may be present as a modified enzyme. Preferably the modified BVMO enzyme is a genetically modified enzyme wherein the genetic material of the BVMO enzyme has been altered from the wild type.

In one embodiment, the genetically modified BVMO enzyme may be a fusion protein which has been constructed from parts of the wild type genetic sequence of one or more of the abovementioned Baeyer-Villiger Monooxygenases so as to create a chimera. Preferred examples of such chimeric BVMOs include, for example: PASTMO (a fusion of PAMO and STMO), or PACHMO (a fusion of PAMO and CHMO) as described by van Beek et al. in Chemical Communications 2012, 48, 3288-3290.

Preferably the Baeyer-Villiger monooxygenase is produced by propagating a host organism which has been transformed with the relevant nucleic acids to express said Baeyer-Villiger monooxygenase in a manner known in the art. Suitable host organisms include, but are not limited to: bacteria, fungi, yeasts, plants, algae, protists, etc.

Preferably the relevant nucleic acids are expressed upon an expression vector within the host organism. Suitable expression vectors include any commercially available vector known in the art, such as, but are not limited to; phage, plasmids, cosmids, phagemid, fosmid, bacterial artifical chromosomes, yeast artificial chromosomes etc.

Suitably the most appropriate vector, method of transformation, and all other associated processes necessary for the expression of a BVMO enzyme in a host organism, as discussed below for bacteria, are adapted for the relevant host organism as known in the art.

Preferably the host organism is a bacterium. Suitably, therefore the expression vector used is any commercially available plasmid, such as, but not limited to: pBR, pUC, pBS, pBE, CoIE, pUT, pACYC, pA, pRAS, pTiC, pBPS, pUO, pKH, pWKS, pCD, pCA, pBAD, pBAC, pMAK, pBL, pTA, pCRE, pHT, pJB, pET, pLME, pMD, pTE, pDP, pSR etc. More preferably the expression vector used is one of the following commercially available plasmids; pBAD, pCRE or pET.

Optionally, the expression vector may be a modified expression vector which is not commercially available and has been altered such that it is tailored to the particular expression of a BVMO enzyme within a host organism. Accordingly, in a preferred embodiment, the expression vector used is the pCRE2 plasmid, based on the commercial pBAD plasmid for expression of the BVMO enzyme in a host bacterium, as described in Torres Pazmino et al. ChemBioChem 10:2595-2598 (2009).

Preferably the host bacterium is transformed by any suitable means known in the art, including, but not limited to; microinjection, ultrasound, freeze-thaw methods, microporation or the use of chemically competent cells. More preferably the host bacterium is transformed by electroporation.

Suitable host bacteria include those from the genus; Streptomyces, Escherichia, Bacillus, Streptococcus, Salmonella, Staphylococcus, or Vibrio. Preferably the host bacterium is selected from the genus Escherichia. More preferably the host bacterium is the species Escherichia coli. Most preferably the host bacterium is the strain Escherichia coli TOP10.

Preferably the relevant nucleic acids expressed upon the expression vector are genetic sequences encoding the Baeyer-Villiger monooxygenase plus any further genetic sequences necessary to effect its expression in a host bacterium as known in the art, such as, but not limited to; promoters, terminators, downstream or upstream effectors, suppressors, activators, enhancers, binding cofactors, initiators, etc.

Preferably the expression vector further comprises genetic sequences encoding at least one expression marker. The expression marker enables the host bacterial cells which have been transformed correctly to be identified. Suitable expression markers include any known in the art, but are not limited to; an antibacterial resistance gene, a pigment producing gene, a pigment inhibiting gene, a metabolic capacity gene, or a metabolic incapacity gene. More preferably the expression marker is an antibacterial resistance gene. Still more preferably the antibacterial resistance gene is an ampicillin resistance gene. Accordingly, only those bacteria able to grow on media containing ampicillin are expressing the vector and have been transformed correctly.

Preferably the expression vector further comprises genetic sequences encoding at least one activator. The activator enables the host bacterial cells which have been transformed to be stimulated to produce the BVMO enzyme at the appropriate times by interaction with an inducer substance. Suitable activator-inducer systems include any known in the art, but particularly the ara operon where L-arabinose is the inducer, or the lac operon where the inducer is allolactose or IPTG.

Optionally, the expression vector may further comprise genetic sequences encoding a tag. Preferably the genetic sequences encoding said tag are operable to be continuously transcribed with the genetic sequences encoding the Baeyer Villiger Monooxygenase enzyme, such that the tag forms a fusion protein with the resulting Baeyer Villiger Monooxygenase enzyme. The tag enables the resulting Baeyer Villiger Monooxygenase enzyme to be purified easily from the host bacterial lysate. Suitable tags include any known in the art, but are not limited to; a His-tag, a GST tag, a MBP tag, or an antibody tag.

Preferably the host bacterium is grown by culturing it in, or on, a suitable media under suitable conditions as known in the art, wherein the media may be a broth or a set gel. Preferably the media contains a source of nutrients, a selective component to select for the presence of the expression marker, and an inducer to induce expression of the expression vector in the bacteria, wherein the selective component and the inducer are specific to the expression vector used. Preferably the media is a broth. More preferably the media is Luria-Bertani broth.

The Baeyer-Villiger monooxygenase may be present in the reaction mixture of the above process in any suitable form known in the art, such as but not limited to; a free cell extract, a synthetic enzyme, or contained within the host organism cells, and these may be located within the reaction mixture in any suitable way known in the art, such as but not limited to; in free form in solution, held upon a membrane, or bound to/within a column.

Preferably the BVMO is present in the reaction mixture at a concentration necessary to produce the maximal amount of alkyl methacrylate capable of being produced at the relative level of dissolved oxygen. Typically, in an industrial situation, about 0.01 to 0.5 moles of O₂ per litre per hour are dissolved into the reaction mixture which is capable of giving about 0.01 to 0.5 moles of alkyl methacrylate per litre per hour. According to a further preferred embodiment of the first aspect of the present invention there is provided a process of producing an alkyl methacrylate comprising the steps of;
(i) converting an alkylisopropenylketone, preferably, methyl isopropenyl ketone to the corresponding alkyl methacrylate by abnormal oxidation using a wild type Baeyer-Villiger monooxygenase enzyme; wherein 0.01 to 0.5 moles of alkyl methacrylate per litre per hour are produced by the Baeyer-Villiger monooxygenase enzyme.

The term ‘about’ indicates a marginal limit of a maximum of 20% above or below the stated value. Preferably within 10% above or below the stated value.

In one embodiment, the Baeyer-Villiger monooxygenase is present in the reaction mixture as a cell extract from the cell it was expressed in, wherein the cell is preferably the host bacterial cell used to produce the BVMO enzyme. The cell extract may be obtained by any suitable means capable of lysing the host bacterial cells, including, but not limited to; sonication, DNAse/lysozyme treatment, freeze-thaw treatment, or alkaline treatment.

Preferably the cell extract is then treated to remove cellular debris before being used as a source of Baeyer-Villiger monooxygenase in the above process. The cell lysate may be treated by any suitable means known in the art, including, but not limited to; filtration, centrifugation, or purification with salts to obtain a cleared cell extract.

Preferably further components are present in the reaction mixture of the above process in order to allow the Baeyer Villiger Monooxygenase enzyme to function correctly. Preferably the further components are; a buffer or pH stat, NADPH, and optionally an NADPH regenerating agent.

Any suitable buffer may be used in the reaction mixture, suitable buffers include, but are not limited to; Tris-HCl, TAPS, Bicine, Tricine, TAPSO, HEPES, TES, MOPS, PIPES, Cacodylate, SSC, or MES. Preferably the buffer used in the reaction mixture is Tris-HCl.

Alternatively, a pH stat may be used to control the pH of the reaction mixture.

Preferably the buffer or the pH stat maintains the reaction mixture at a suitable pH for the BVMO enzyme to function and/or the host organism comprising said BVMO enzyme to live. Preferably the buffer or the pH stat maintains the reaction mixture at a pH between about pH 6.5 to pH 8.5. More preferably the buffer or the pH stat maintains the reaction mixture at a pH of between about pH 7.3 and 7.7. Still more preferably the buffer or the pH stat maintains the reaction mixture at a pH of about 7.5.

The term ‘about’ as used with reference to the pH of the reaction mixture indicates a marginal limit of a maximum of 20% above or below the stated value. However, preferably the pH of the reaction mixture is within 10% above or below the stated value.

Preferably the concentration of buffer in the reaction mixture is between about 25 to 100 mM. More preferably the concentration of buffer in the reaction mixture is between about 40 and 60 mM. Still more preferably the concentration of buffer in the reaction mixture is about 50 mM.

The term ‘about’ as used with reference to the concentration of buffer indicates a marginal limit of a maximum of 20% above or below the stated value. However, preferably the concentration of buffer is within 10% above or below the stated value.

Preferably the NADPH is present in the reaction mixture at a starting molar concentration relative to BVMO such that the BVMO enzyme is saturated with NADPH. Therefore, preferably the NADPH is present in the reaction mixture at a concentration which is at least equal to the concentration of BVMO enzyme.

Preferably in one embodiment, the NADPH is present in the reaction mixture at a starting concentration of between about 50 to 200 μM. More preferably the NADPH is present in the reaction mixture at a starting concentration of between about 90 to 110 μM. Still more preferably the NADPH is present in the reaction mixture at a starting concentration of about 100 uM.

The term ‘about’ as used with reference to the concentration of NADPH indicates a marginal limit of a maximum of 20% above or below the stated value. However, preferably the concentration of NADPH is within 10% above or below the stated value.

Preferably the NADPH regenerating agent is present in the reaction mixture at a concentration of between about 5 to 20 μM. More preferably the NADPH regenerating agent is present in the reaction mixture at a concentration of between about 8 to 12 uM. Still more preferably the NADPH regenerating agent is present in the reaction mixture at a concentration of about 10 uM.

Preferably, if used, the NADPH regenerating agent is present in the reaction mixture at a molar concentration relative to BVMO such that the BVMO is saturated with NADPH. Preferably therefore, the Km of the NADPH regenerating agent, if used, is at least equivalent to the rate of consumption of NADPH by the BVMO.

The term ‘about’ as used with reference to the concentration of NADPH regenerating agent indicates a marginal limit of a maximum of 20% above or below the stated value. However, preferably the concentration of NADPH regenerating agent is within 10% above or below the stated value.

Any suitable NADPH regenerating agent may be used in the reaction mixture, such as, but not limited to; phosphite dehydrogenase, glucose-6-phosphate dehydrogenase, alcohol dehydrogenase, or formate dehydrogenase. Suitably, the relevant partner substrate to the NADPH regenerating agent is also present within the reaction mixture, such as, but not limited to; glucose, an alcohol, phosphite or formate.

Alternatively, NADPH may be provided into the reaction mixture as a macromolecular cofactor covalently linked to a support, for example a membrane, resin, or gel.

Preferably the partner substrate is present in the reaction mixture at a concentration of between 5 mM to 20 mM, more preferably at a concentration of between about 8 mM to 12 mM, still more preferably at a concentration of about 10 mM.

Preferably the partner substrate is present in the reaction mixture at a molar concentration relative to the NADPH regenerating agent (NADPH regenerating agent: partner substrate) of between about 1:4000 and 1:250. More preferably the partner substrate is present in the reaction mixture at a molar concentration relative to the NADPH regenerating agent of about 1:1000.

The term ‘about’ as used with reference to the concentration of partner substrate to the NADPH regenerating agent indicates a marginal limit of a maximum of 20% above or below the stated value. However, preferably the concentration of partner substrate to the NADPH regenerating agent is within 10% above or below the stated value.

Suitably, substrate is present in the above reaction mixture in order to start the BVMO conversion of alkylisopropenyl ketone to alkyl methacrylate. Preferably, in such an embodiment, the concentration of alkylisopropenylketone substrate present in the above reaction mixture is between about 10 g/L and 200 g/L. More preferably the concentration of alkylisopropenylketone substrate present in the above reaction mixture is between about 50 g/L and 130 g/L. Still more preferably the concentration of alkylisopropenylketone substrate present in the above reaction mixture is between about 90 g/L and 110 g/L

The term ‘about’ as used with reference to the concentration of substrate indicates a marginal limit of a maximum of 20% above or below the stated value. However, preferably the concentration of substrate is within 10% above or below the stated value.

Preferably, in such an embodiment, the concentration of alkylisopropenylketone substrate present in the above reaction mixture is at least about 10% by weight of the reaction mixture, more preferably it is between at least about 20% by weight of the reaction mixture, up to about 80% by weight of the reaction mixture.

In an alternative embodiment, the Baeyer-Villiger monooxygenase may be present in the reaction mixture as a synthetic enzyme. In such an embodiment, the synthetic enzyme is synthesised in vitro in a manner known in the art then purified before being used in the reaction mixture. Preferably the reaction mixture comprises the same components as defined in the reaction mixture above at substantially the same concentrations and ratios.

In a further alternative embodiment, the Baeyer-Villiger Monooxygenase may be present in the reaction mixture within the host organism cells, such as bacterial cells which feature may be combined with any of the aspects, embodiments, or other preferred features of the present invention as contained herein.

According to a further preferred embodiment of the first aspect of the present invention there is provided a process of producing an alkyl methacrylate comprising the steps of;
(i) converting an alkylisopropenylketone, preferably, methyl isopropenylketone to the corresponding alkyl methacrylate by abnormal oxidation using a Baeyer-Villiger monooxygenase enzyme in a reaction mixture, wherein the Baeyer-Villiger monooxygenase enzyme is present in the reaction mixture within host organism cells.

In such an embodiment, the host cells are prepared in a manner known in the art then purified before being used in the reaction mixture. Preferably the reaction mixture comprises buffer and substrate as defined in the reaction mixture above. Preferably the buffer is present at the same concentrations and ratios defined above.

However, preferably, in such an embodiment, the concentration of alkylisopropenylketone substrate present in the above reaction mixture is less than the concentration limit which is toxic to the host cells, and which is optimal for uptake of the substrate into the host cells which concentration of alkylisopropenylketone substrate may be combined with any of the aspects, embodiments, or other preferred features of the present invention as contained herein which are capable of working with the Baeyer-Villiger Monooxygenase being present in the reaction mixture within the host organism cells.

Preferably, therefore, the concentration of alkylisopropenylketone substrate present in the above reaction mixture is between about 0.2 g/L and 50 g/L. More preferably the concentration of alkylisopropenylketone substrate present in the above reaction mixture is between about 0.2 g/L and 30 g/L. Still more preferably the concentration of alkylisopropenylketone substrate present in the above reaction mixture is between about 0.2 g/L and 20 g/L.

The term ‘about’ as used with reference to the concentration of substrate indicates a marginal limit of a maximum of 20% above or below the stated value. However, preferably the concentration of substrate is within 10% above or below the stated value.

Preferably, in such an embodiment, the concentration of alkylisopropenylketone substrate present in the above reaction mixture is at least about 1% by weight of the reaction mixture, more preferably it is between at least about 2% by weight of the reaction mixture, up to about 20% by weight of the reaction mixture.

Suitably, in such an embodiment, the concentration of BVMO enzyme present in the reaction mixture is determined by the concentration of host cells present in the reaction media. Preferably the concentration of host cells present in the reaction media is between about 1 g/L and 100 g/L. More preferably the concentration of host bacterial cells present in the reaction media is between about 5 g/L and 50 g/L. Still more preferably the concentration of host bacterial cells present in the reaction media is between about 10 g/L and 20 g/L. Typically, the host cells are bacterial cells.

According to a further preferred embodiment of the first aspect of the present invention there is provided a process of producing an alkyl methacrylate comprising the steps of;
(i) converting an alkylisopropenylketone, preferably, methyl isopropenylketone to the corresponding alkyl methacrylate by abnormal oxidation using a Baeyer-Villiger monooxygenase enzyme present within host bacterial cells.

Preferably the host bacterial cells are as defined above in relation to the production of the Baeyer-Villiger monooxygenase enzyme, preferably the Baeyer-Villiger monooxygenase enzyme is produced in the same host bacterial cell which is used in the process of the invention.

Suitable host bacteria include those from the genus; Streptomyces, Escherichia, Bacillus, Streptococcus, Salmonella, Staphylococcus, or Vibrio.

Preferably the host bacterium is selected from the genus Escherichia.

More preferably the host bacterium is the species Escherichia coli. Most preferably the host bacterium is the strain Escherichia coli TOP10.

According to a further preferred embodiment of the first aspect of the present invention there is provided a process of producing an alkyl methacrylate comprising the steps of;
(i) converting an alkylisopropenylketone, preferably, methyl isopropenylketone, to the corresponding alkyl methacrylate by abnormal oxidation using a Baeyer-Villiger monooxygenase enzyme present within Escherichia coli cells.

Preferably the relevant nucleic acids are expressed upon an expression vector within the host organism.

Suitable expression vectors include any commercially available vector known in the art, such as, but are not limited to; phage, plasmids, cosmids, phagemid, fosmid, bacterial artifical chromosomes, yeast artificial chromosomes etc.

Preferably the host organism is a bacterium.

Suitably, therefore the expression vector used is any commercially available plasmid, such as, but not limited to: pBR, pUC, pBS, pBE, CoIE, pUT, pACYC, pA, pRAS, pTiC, pBPS, pUO, pKH, pWKS, pCD, pCA, pBAD, pBAC, pMAK, pBL, pTA, pCRE, pHT, pJB, pET, pLME, pMD, pTE, pDP, pSR etc.

More preferably the expression vector used is one of the following commercially available plasmids; pBAD, pCRE or pET.

Optionally, the expression vector may be a modified expression vector which is not commercially available and has been altered such that it is tailored to the particular expression of a Baeyer-Villiger monooxygenase enzyme within a host organism. Accordingly, in a preferred embodiment, the expression vector used is the pCRE2 plasmid, based on the commercial pBAD plasmid for expression of the BVMO enzyme in a host bacterium, as described in Torres Pazmino et al. ChemBioChem 10:2595-2598 (2009).

Suitably in such an embodiment, the reaction mixture does not comprise added NADPH or an optional NADPH regenerating agent and partner substrate because they are already present within the host cell biochemistry.

Preferably, regardless of the form of BVMO source used in the reaction mixture, an in situ product removal system is implemented together with a substrate feeding strategy in the reaction process. It has been found that removal of product together with a constant substrate feed can increase product yields to much higher values, as described by Alphand et al. in Trends in Biotechnology Vol. 21 No. 7 Jul. 2003. Any product removal system and any substrate feeding strategy known in the art may be implemented. However, preferably the product removal system and substrate feeding system are implemented using the same technology, for example, by the use of a carrier material which can simultaneously act as a reservoir for substrate and a sink for product. One such technology is the use of Optipore L-493 resin described by Simpson et al. Journal of Molecular Catalysis B Enzyme 16, pp. 101-108.

The term ‘absolute level’ as used herein refers to the actual percentage value of the alkyl methacrylate obtained as a product in solution from the conversion of the alkylisopropenylketone. The term ‘relative level’ as used herein refers to the selectivity i.e. the percentage of alkyl methacrylate obtained as a product in solution compared to the alternative product isopropenylester obtained as a product in solution from the conversion of alkylisopropenylketone.

Preferably, therefore, the ratio of alkyl methacrylate:isopropenylester production by the BVMO enzyme in the above process is at least 1:5, more preferably at least 1:2, still more preferably at least 1:1.5, most preferably at least 1:0.5 which ratios may be selected and combined with any of the aspects, embodiments, or other preferred features of the present invention as contained herein.

According to a further preferred embodiment of the first aspect of the present invention there is provided a process of producing an alkyl methacrylate comprising the steps of;
(i) converting an alkylisopropenylketone, preferably, methyl isopropenylketone, to the corresponding alkyl methacrylate by abnormal oxidation using a wild type Baeyer-Villiger monooxygenase enzyme; wherein the ratio of alkyl methacrylate:isopropenylester production by the Baeyer-Villiger monooxygenase enzyme is at least 1:5

Preferably, therefore, the Baeyer Villiger monooxygenase converts the alkylisopropenylketone to the alkyl methacrylate at an absolute level of at least 1% selectivity in the above process. More preferably the Baeyer Villiger monooxygenase enzyme converts the alkylisopropenylketone to the alkyl methacrylate at an absolute level of at least 2% selectivity. Still more preferably the Baeyer Villiger monooxygenase converts the alkylisopropenylketone to the alkyl methacrylate at an absolute level of at least 5% selectivity which selectivities may be combined with any of the aspects, embodiments, or other preferred features of the present invention as contained herein.

According to a further preferred embodiment of the first aspect of the present invention there is provided a process of producing an alkyl methacrylate comprising the steps of;
(i) converting an alkylisopropenylketone, preferably, methyl isopropenylketone, to the corresponding alkyl methacrylate by abnormal oxidation using a wild type Baeyer-Villiger monooxygenase enzyme; wherein the Baeyer Villiger monooxygenase converts the alkylisopropenylketone to the alkyl methacrylate at an absolute level of at least 1% selectivity

Preferably the Baeyer Villiger Monooxygenase converts the alkylisopropenylketone to the alkyl methacrylate at a relative level of at least 20%, more preferably the Baeyer Villiger monooxygenase enzyme converts the alkylisopropenylketone to the alkyl methacrylate at a relative level of at least 50%, still more preferably the Baeyer Villiger monooxygenase enzyme converts the alkylisopropenylketone to the alkyl methacrylate at a relative level of at least 80%, especially, at least 90%, for example 98 or 99% which relative levels may be selected and combined with any of the aspects, embodiments, or other preferred features of the present invention as contained herein.

According to a further preferred embodiment of the first aspect of the present invention there is provided a process of producing an alkyl methacrylate comprising the steps of;
(i) converting an alkylisopropenylketone, preferably, methyl isopropenylketone to the corresponding alkyl methacrylate by abnormal oxidation using a wild type Baeyer-Villiger monooxygenase enzyme; wherein the Baeyer Villiger Monooxygenase converts the alkylisopropenylketone to the alkyl methacrylate at a relative level of at least 20%

According to a second aspect of the present invention there is provided a method of preparing polymers or copolymers of an alkyl methacrylate comprising the steps of:
(i) preparation of an alkyl methacrylate in accordance with the first aspect of the present invention;
(ii) optionally, transesterifying the alkyl methacrylate to produce a transesterified alkyl methacrylate
(ii) polymerisation of the alkyl methacrylate or transesterified alkyl methacrylate prepared in (i) or (ii), optionally with one or more comonomers, to produce polymers or copolymers thereof.

Therefore, according to a particularly preferred embodiment of a second aspect of the present invention, there is provided a method of preparing polymers or copolymers of methyl methacrylate comprising the steps of:
(i) preparation of methyl methacrylate in accordance with the first aspect of the present invention;
(ii) polymerisation of the methyl methacrylate prepared in (i), optionally with one or more comonomers, to produce polymers or copolymers thereof.

Therefore, according to a third aspect of the present invention, there is provided a method of preparing polymers or copolymers of ethyl methacrylate comprising the steps of:
(i) preparation of ethyl methacrylate in accordance with the first aspect of the present invention;
(ii) polymerisation of the ethyl methacrylate prepared in (i), optionally with one or more comonomers, to produce polymers or copolymers thereof.

Therefore, according to a fourth aspect of the present invention, there is provided a method of preparing polymers or copolymers of a transesterified alkyl methacrylate comprising the steps of:
(i) preparation of alkyl methacrylate in accordance with the first aspect of the present invention;
(ii) transesterifying the alkyl methacrylate to produce a transesterified alkyl methacrylate;
(iii) polymerisation of the transesterified alkyl methacrylate prepared in (ii), optionally with one or more comonomers, to produce polymers or copolymers thereof.

Advantageously, such polymers will have an appreciable portion if not all of the monomer residues derived from a renewable biomass source other than fossil fuels.

Preferably, the alkyl methacrylate is selected from either methyl methacrylate or ethyl methacrylate and the transesterified alkyl methacrylate is prepared from the alkyl methacrylate by transesterification with a suitable alcohol. Examples of transesterified alkyl methacrylates include ethyl methacrylate, n-butyl methacrylate, iso-butyl methacrylate, t-butyl methacrylate, 2-ethylhexyl methacrylate, lauryl methacrylate, cyclohexyl methacrylate, isobornyl methacrylate, 2-hydroxyethyl methacrylate and hydroxypropylethyl methacrylate, phenoxyethyl methacrylate, hexadecyl methacrylate. More preferably, the alkyl methacrylate is methyl methacrylate. Preferred examples of transesterified alkyl methacrylates are n-butyl and iso-butyl methacrylate.

Suitable alcohols for transesterifying the alkyl methacrylate include C3-C18 alcohols which may be linear or branched, aliphatic, aromatic, cyclic or part cyclic or part aromatic and optionally substituted with an hydroxyl, halo, epoxy or amino group and/or be interrupted by hetero atoms such as oxygen. Preferred alcohols correspond to the transesterified alkyl methacrylate examples above, preferred alcohols are those which can be made from a biomass source, for example 1-butanol or 2-methyl-1-propanol. For the purpose of this definition, alkyl may be taken to mean

In any case, preferred comonomers include for example, monoethylenically unsaturated carboxylic acids and dicarboxylic acids and their derivatives, such as esters, amides and anhydrides.

Particularly preferred comonomers are acrylic acid, methyl acrylate, ethyl acrylate, propyl acrylate, n-butyl acrylate, iso-butyl acrylate, t-butyl acrylate, 2-ethylhexyl acrylate, hydroxyethyl acrylate, iso-bornyl acrylate, methacrylic acid, ethyl methacrylate (in relation to the second or fourth aspect) or methyl methacrylate (in relation to the third or fourth aspect), propyl methacrylate, n-butyl methacrylate, iso-butyl methacrylate, t-butyl methacrylate, 2-ethylhexyl methacrylate, hydroxyethyl methacrylate, lauryl methacrylate, glycidyl methacrylate, hydroxypropyl methacrylate, iso-bornyl methacrylate, dimethylaminoethyl methacrylate, tripropyleneglycol diacrylate, styrene, α-methyl styrene, vinyl acetate, isocyanates including toluene diisocyanate and p,p′-methylene diphenyl diisocyanate, acrylonitrile, butadiene, butadiene and styrene (MBS) and ABS.

According to a fifth aspect of the present invention there is provided a polyalkylmethacrylate homopolymer or copolymer formed from the method according to the second aspect of the present invention.

Preferably the polyalkylmethacrylate is one of polymethylmethacrylate or polyethylmethacrylate. More preferably the polyalkylmethacrylate is polymethylmethacrylate.

Therefore, according to a particularly preferred embodiment of a fifth aspect of the present invention, there is provided a polymethylmethacrylate homopolymer or copolymer formed from the method according to the second aspect of the present invention.

Therefore, according to a sixth aspect of the present invention, there is provided a polyethylmethacrylate homopolymer or copolymer formed from the method according to the third aspect of the present invention.

Furthermore, according to a seventh aspect of the present invention, there is provided a polytransesterified alkyl methacrylate homopolymer or copolymer formed from the method according to the fourth aspect of the present invention.

Advantageously the present invention provides a process for the production of MMA and derivatives thereof by the use of a Baeyer Villiger Monooxygenase enzyme to catalyse the abnormal conversion of an aliphatic alkylisopropenylketone to the relevant alkyl acrylate at an industrially applicable level.

All of the features contained herein may be combined with any of the above aspects, in any combination which allows the formation of an alkylisopropenylketone for conversion to an alkyl methacrylate using a BVMO enzyme.

The terms may be selected and combined with any of the aspects, embodiments, or other preferred features of the present invention as contained herein or the like does not extend to combinations where at least some of such features and/or steps are mutually exclusive.

For a better understanding of the invention, and to show how embodiments of the same may be carried into effect, reference will now be made, by way of example, to the following figures and examples in which:—

### EXAMPLE 1

### Chemicals and Enzymes

All chemicals were of analytical grade and obtained from Sigma Aldrich. The genes encoding various BVMO enzymes reported in the literature were obtained and expressed in E. coli fused to the N-terminus of a thermostable phosphite dehydrogenase (PTDH, EC 1.20.1.1) for cofactor regeneration. The host E. coli cells were then lysed by sonication to obtain disrupted cell fractions, and underwent centrifugation to remove cell debirs and obtain a cleared cell extract. See methods as described in ‘Expanding the set of rhodococcal Baeyer Villiger Monooxygenases by high throughput cloning, expression and substrate screening’ by Riebel et al. Appl. Microbiol. Biottechnol. (2011).

Biocatalysis Protocol

Transformations were performed in 15 ml Pyrex tubes. Reaction volumes (1 ml) contained 5 mM methylisopropenylketone, 5 uM ‘reducing FAD’, and 5 μM cleared cell extract containing the relevant BVMO, in 50 mM Tris-HCl, pH 7.5. Mixtures were incubated at 24° C. under orbital shaking (200 rpm) for 22 hours. To determine conversion, 1 ml reaction volume was extracted with 0.5 ml 1-octanol containing 0.1% mesitylene (1,3,5-trimethylbenzene) as internal standard. Samples were extracted by vortexing for 1 min, followed by a centrifugation step (5000 rpm) for 10 min. The organic layer was removed, dried with MgSO₄ and placed in a gas chromatography (GC) vial. GC analysis occurred on a Shimadzu GC instrument fitted with a Heliflex® AT™-5 column (Grace Discovery Sciences). The following temperature profile was used to separate the components: 6 min at 40° C. followed by an increase to 250° C. at 20° C. per minute. Blank reactions without enzyme and with varying amounts of substrate (methylisopropenylketone) and product (methyl methacrylate, isopropenyl acetate) were carried out under identical circumstances and used to prepare calibration curves for product identification and determination of conversion.

GC Analysis

Table 1 below details the percentage of the compounds produced by GC following extraction with 1-octanol+0.1% mesitylene from 50 mM Tris-HCl, pH 7.5 (AT-5 column, 5 mM all compounds). All three compounds (1 substrate and 2 products) could reliably be separated by GC.

TABLE 1

| | isopropenyl | methyl |
|---|---|---|
| BVMO | acetate | methacrylate |
| CHMO | 0 | ~1% |
| Rhodococcus josti | | |
| RHA1 | | |

Table 1 shows that the inventors have discovered that BVMO enzymes act to produce an abnormal oxygen insertion on the substrate methylisoproenylketone to give a yield of 1% methyl methacrylate product, and furthermore which do so exclusively of the expected normal productof isopropenyl acetate. The BVMO enzyme in this example is a cyclohexanone monooxygenase enzyme (CHMO) accession number ro06679, produced natively by the organism Rhodococcus jostii RHA1.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A process of producing an alkyl methacrylate comprising the step of converting an alkylisopropenylketone to the corresponding alkyl methacrylate using a Baeyer-Villiger monooxygenase enzyme.

2. The process according to claim 1, wherein the conversion of the alkylisopropenylketone to the corresponding alkyl methacrylate is by abnormal oxidation using a Baeyer-Villiger monooxygenase enzyme.

3. The process according to claim 1, wherein the alkylisopropenylketone is methylisopropenylketone or ethylisopropenylketone, and the alkyl methacrylate produced is respectively methyl methacrylate or ethyl methacrylate.

4. The process according to claim 1, wherein the Baeyer-Villiger monooxygenase enzyme is a wild type enzyme or a modified enzyme.

5. The process according to claim 1, wherein the Baeyer-Villiger monooxygenase enzyme is a wild type enzyme.

6. The process according to claim 1, wherein the Baeyer-Villiger monooxygenase enzyme is a modified enzyme.

7. The process according to claim 1, further comprising the step of formation of an alkylisopropenylketone from raw feedstocks.

8. The process according to claim 7, further comprising the step of performing one or more chemical, biochemical or biological conversions to produce the raw feedstocks.

9. The process according to claim 7, wherein the raw feedstocks include 2-butanone, methylisopropylketone, 3-pentanone, ethylisopropylketone and formaldehyde or a derivative thereof.

10. The process according to claim 9, wherein the modified Baeyer-Villiger monooxygenase enzyme is as active and/or selective as the wild type in the oxidative transformation of the alkylisopropenylketone to produce alkyl methacrylate.

11. The process according to claim 1, wherein the Baeyer-Villiger monooxygenase enzyme is sourced from a bacterium optionally selected from the following bacterial genera Acinetobacter, Rhodococcus, Arthrobacter, Brachymonas, Nocardia, Exophiala, Brevibacterium, Gordonia, Novosphingobium, Streptomyces, Thermobifida, Xanthobacter, Mycobacterium, Comamonas, Thermobifida and Pseudomonas.

12. The process according to claim 1, wherein the Baeyer-Villiger monooxygenase enzyme is sourced from a fungus optionally selected from the following fungal genera Gibberella, Aspergillus, Maganporthe, Cylindrocarpon, Curvularia, Drechslera, Saccharomyces, Candida, Cunninghamella, Cylindrocarpon, and Schizosaccharomyces.

13. The process according to claim 1, wherein the Baeyer-Villiger monooxygenase enzyme is sourced from an archaeon.

14. The process according to claim 1, wherein the Baeyer-Villiger monooxygenase is a type I, type II or type O Baeyer-Villiger monooxygenase.

15. The process according to claim 1, wherein the Baeyer-Villiger monooxygenase is a type I Baeyer-Villiger monooxygenase selected from one of the following enzyme groups: a cyclohexanone monoxygenase, a 4-hydroxyacetophenone monooxygenase, a cyclopentadecanone monooxygenase or an acetone monoxygenase.

16. The process according to claim 1, wherein the Baeyer-Villiger monooxygenase is selected from: a type I Baeyer-Villiger monooxygenase selected from one of the following enzyme groups; a cyclohexanone monooxygenases (CHMO) EC number 1.14.13.22 (GenBank: BAA86293.1); a phenylacetone monooxygenases (PAMO) EC number 1.14.13.92 (Swiss-Prot: Q47PU3); a 4-hydroxyacetophenone monooxygenase (HAPMO) EC number 1.14.13.84 (GenBank: AAK54073.1); an acetone monooxygenases (ACMO) (GenBank: BAF43791.1); a methyl ketone monooxygenases (MEKA) (GenBank: ABI15711.1); a cyclopentadecanone monooxygenases (CPDMO) (GenBank: BAE93346.1); a cyclopentanone monooxygenases (CPMO) (GenBank: BAC22652.1); and a steroid monooxygenases (STMO) (GenBank: BAA24454.1).

17. The process according to claim 1, wherein the Baeyer-Villiger monooxygenase is a cyclohexanone monoxygenase, a 4-hydroxyacetophenone monooxygenase, a cyclopentadecanone monooxygenase or an acetone monoxygenase, which may optionally be selected from one of the following enzymes: cyclohexanone monooxygenase from Acinetobacter calcoaceticus NCIMB 9871, cyclohexanone monooxygenase from Xanthobacter flavus (GenBank: CAD10801.1), cyclohexanone monooxygenase from Rhodococcus sp. HI-31 (GenBank: BAH56677.1), cyclohexanone monooxygenase from Rhodococcus jostii RHA1, cyclohexanone monoxygenase from Brachymonas petroleovorans (GenBank: AAR99068.1), 4-hydroxyacetophenone monooxygenase (Q93TJ5.1), cyclopentadecanone monooxygenase (GenBank: BAE93346.1), or acetone monooxygenase from Gordonia sp. TY-5 (Genbank: BAF43791.1).

18. The process according to claim 1, wherein the Baeyer-Villiger monooxygenase enzyme is present in a reaction mixture of the above process as a free cell extract, or a synthetic enzyme.

19. The process according to claim 18 wherein the concentration of alkylisopropenylketone substrate present in the reaction mixture is between about 10 g/L and 200 g/L.

20. The process according to claim 18, wherein the concentration of alkylisopropenylketone substrate present in the reaction mixture is at least about 10% by weight of the reaction mixture.

21. The process according to claim 1, wherein the Baeyer-Villiger monooxygenase enzyme is present in the reaction mixture within host organism cells.

22. The process according to claim 21, wherein the concentration of alkylisopropenylketone substrate present in the above reaction mixture is less than the concentration limit which is toxic to host cells.

23. The process according to claim 22, wherein the concentration of alkylisopropenylketone substrate present in the above reaction mixture is between about 0.2 g/L and 50 g/L.

24. The process according to claim 22, wherein the concentration of alkylisopropenylketone substrate present in the above reaction mixture is at least about 1% by weight of the reaction mixture.

25. The process according to claim 21, wherein the concentration of host cells present in the reaction mixture is between about 1 g/L and 100 g/L.

26. The process according to claim 21, wherein the Baeyer-Villiger monooxygenase enzyme is present within host bacterial cells or alternatively, host fungal cells or alternatively archaeon cells.

27. The process according to claim 1, wherein an in situ product removal system is implemented together with a substrate feeding strategy in the reaction process.

28. The process according to claim 1, wherein the ratio of alkyl methacrylate:isopropenylester production by the Baeyer Villiger monoxygenase enzyme is at least 1:5.

29. The process according to claim 1, wherein the Baeyer Villiger monoxygenase enzyme converts the alkylisopropenylketone to the alkyl methacrylate at an absolute level of at least 1% selectivity.

30. The process according to claim 1, wherein the Baeyer Villiger Monooxygenase enzyme converts the alkylisopropenylketone to the alkyl methacrylate at a relative level of at least 20%.

31. A method of preparing polymers or copolymers of an alkyl methacrylate comprising the steps of:
preparing an alkyl methacrylate according to claim 1;
optionally, transesterifying the alkyl methacrylate to produce a transesterified alkyl methacrylate;
polymerizing the alkyl methacrylate or transesterified alkyl methacrylate, optionally with one or more comonomers, to produce polymers or copolymers thereof.

32. The method according to claim 31, wherein the alkyl methacrylate is selected from methyl methacrylate and ethyl methacrylate.

33. The method according to claim 31, wherein the transesterified alkyl methacrylate is prepared from the alkyl methacrylate by transesterification with a suitable alcohol.

34. The method according to claim 31, wherein the transesterified alkyl methacrylate is selected from ethyl methacrylate, n-butyl methacrylate, iso-butyl methacrylate, t-butyl methacrylate, 2-ethylhexyl methacrylate, lauryl methacrylate, cyclohexyl methacrylate, isobornyl methacrylate, 2-hydroxyethyl methacrylate and hydroxypropylethyl methacrylate, phenoxyethyl methacrylate, hexadecyl methacrylate.

35. The method according to claim 33, wherein the alcohol is selected from a C3-C18 alcohol which may be linear or branched, aliphatic, aromatic, cyclic or part cyclic or part aromatic and optionally substituted with an hydroxyl, halo, epoxy or amino group and/or be interrupted by hetero atoms.

36. The method according to claim 31, wherein the comonomer is selected from monoethylenically unsaturated carboxylic acids and dicarboxylic acids and their derivatives.

37. A polyalkylmethacrylate homopolymer or copolymer formed from the method according to claim 31.
